(19) United States
(12) Patent Application Publication
(10) Pub. No.: US 2026/0328744 A1
(43) Pub. Date: Oct. 22, 2026

(54) Methods and Systems for Engine and Emission Monitoring and Modeling

(71) Applicant: TS Cyanergy Inc., Houston, TX (US)

(72) Inventors: Steven Manz, Sugar Land, TX (US); Mohammed Khambaty, Katy, TX (US); Roy McIntyre, Temple, TX (US); Timothy J. Jacobs, College Station, TX (US)

(21) Appl. No.: 19/648,532

(22) Filed: Apr. 15, 2026

Related U.S. Application Data
(60) Provisional application No. 63/789,636, filed on Apr. 16, 2025.

(51) Int. Cl. G01N 33/00; G07C 5/00

(52) U.S. Cl. CPC G01N 33/0073; G07C 5/008

(57) ABSTRACT

In one embodiment, a system includes an engine and emission monitor having multiple connection ports supporting different data communication protocols and a wireless transceiver configured for long-range wide area network communications. One or more processors are operably coupled to the engine and emission monitor. An exhaust gas analyzer is coupled to the engine and emission monitor and to exhaust pipes of engines. The exhaust gas analyzer measures concentration values of gases in exhaust gas and transmits the measured concentration values to at least one of the processors. At least one processor executes a stoichiometric model to convert the measured concentration values into corresponding mass values based on an approximate molecular weight and a mass value of the exhaust gas and molecular weights of the measured gases. The approximate molecular weight of the exhaust gas is calculated by summing products of each gas concentration value and its molecular weight.

### PRIORITY

This application claims the benefit, under 35 U.S.C. § 119(e), of U.S. Provisional Patent Application No. 63/789,636, filed Apr. 16, 2026, which is incorporated herein by reference in its entirety.

### TECHNICAL FIELD

This disclosure generally relates to engine and emission monitoring, calculation, and modeling.

### BACKGROUND

Large ships contribute about 4% of global greenhouse gas (GHG) emissions, including CO₂, CH₄, and N₂O. Emissions from inland vessels (e.g., tugboats and barges) represent approximately 3% of global GHG emissions. Emissions from upstream oil and gas activities (e.g., drilling, fracturing, and production), which include drilling rigs, trucks, fracking units, boats, compression systems, etc., represent approximately 5% of global GHG emissions.

The shipowners and oil and gas companies are required to report at least a portion of their emissions. For example, European companies must report scope 1, 2 and 3 emissions. The International Maritime Organization requires all ocean bound ships must report their emissions. Ships which operate in Europe must purchase carbon allowances, which are purchased at one of the regulated carbon exchanges. The prices fluctuate, and currently the price is approximately €60/ton of CO2e. Inland vessels are required to report certain gases to the EPA and the EEA (EU comparable regulatory to the EPA).

There are several acceptable methods of measuring the emissions, but the common ones include the calculate method and direct measurement method. For the calculated method, the reported emissions are estimated based on the actual fuel consumption and a standard conversion factor table published by the varies regulatory bodies (e.g., EPA). For the direct measurement method, the emissions are measured from sensors in the actual exhaust pipes which sample the mass of the GHG.

### BRIEF DESCRIPTION OF THE DRAWINGS

FIG. 1A illustrates an example engine and emission monitor in accordance with certain embodiments of the present disclosure.

FIG. 1B illustrates an example engine and emission monitor connected to an engine in accordance with certain embodiments of the present disclosure.

FIG. 2 an example flow diagram for measuring and calculating emissions using methods and systems in accordance with certain embodiments of the present disclosure.

FIG. 3 illustrates a computer system in accordance with certain embodiments of the present disclosure.

### DESCRIPTION OF EXAMPLE EMBODIMENTS

### Overview

The mass of the GHG in the exhaust may be a function of factors including type of fuel, engine efficiency, airflow, and engine status. Type of fuel may be impacted by the use of additives or alternative fuels (e.g., ammonia). With respect to engine efficiency (e.g., power loads), engines operating at a lower or higher power may emit more GHG. Higher airflow may reduce the GHG from a constant fuel consumption. With respect to engine status, engines that were idle may emit more GHG than engines operating in steady state. For example, the engine may burn more fuel before it is warmed up.

The calculated method for measuring GHG in conventional systems may be inherently inaccurate. The standard conversion factors may be based on the average fuel consumption, i.e., mass of the emissions in engines running at optimal efficiency. The calculated method may not take into account additional factors such as the size or type of engines, the fuel being consumed, the airflow rate, the power used to produce the exhaust, etc.

The direct measurement method for measuring GHG in conventional systems may be more accurate than the calculated method. Direct measurement may require the use of an exhaust gas analyzer. Direct measurement may incorporate all the factors including the size or type of engines, the fuel being consumed, the airflow rate, the power used to produce the exhaust, but the equipment and installation costs can be significant. Additionally, most exhaust gas analyzers may require “purging” the exhaust and particles from the system or components thereof to ensure a clean measurement. When the system is purging, the system may inject clean air (e.g., oxygen) to flush residual carbon dioxide, methane, etc. in order to clean the system or components thereof (e.g., sensors). The purging process may occur frequently at various intervals (e.g., every 4-6 hours) and last several minutes (e.g., 10-12) minutes. As a result, there may be a period of time (e.g., from about 40 minutes to about 72 minutes) every day that the data collected from an exhaust gas analyzer is not reliable.

In particular embodiments, an engine and emission monitor based on wireless Long Range Wide Area Network (LoRaWAN) technology can automatically measure emissions (e.g., greenhouse gases) from engines, such as those used on large ships, inland vessels, and upstream oil and gas activities. Although referred to herein as “engine and emission monitor,” the monitor may be used to monitor only one or more engines, only emissions from one or more engines, and/or both as described herein. The engine and emission monitor can read data from the engines directly. the engine and emission monitor can collect several measurements, including, but not limited to, the fuel flow, airflow, engine load (i.e., power), engine efficiency, and engine speed (e.g., RPM). The engine and emission monitor can also read other engine pieces data (e.g., temperature, oil pressure, etc.). In particular embodiments, the engine and emission monitor can upload data from multiple types of engines, each of which may have different data interfaces. In certain embodiments, the engine and emissions monitor may be an Internet of Things (IoT) monitor. In such embodiments, the engine and emission monitor can accommodate different firmware. In particular embodiments, the engine and emission monitor can also share data from other machine equipped with LoRaWAN capabilities. For example, the engine and emission monitor can share data between engines and/or pumps in a fracking spread.

In particular embodiments, the engine and emission monitor may be used with one or more exhaust gas analyzers to directly measure emissions from one or more engines. In some embodiments, a manifold device may be used to collect emission measurements for multiple engines through a single exhaust gas analyzer. In such embodiments, when an engine is not being used, the manifold device may close a valve to eliminate data from that engine being measured by the exhaust gas analyzer. In certain embodiments, the engine and emission monitor may be used together with stochiometric models to determine mass of emissions. The data collected by the engine and emission monitor may be input to the stochiometric models, which may calculate the mass of emissions based on such data. In other embodiments, a virtual gas analyzer can be generated using data collected by the engine and emission monitor and one or more machine learning models. In such embodiments, the virtual gas analyzer and/or the one or more machine learning models may use a stoichiometric model of the present disclosure. In such embodiments, the virtual gas analyzer may replace at least the one or more exhaust gas analyzers.

According to an embodiment, a system includes an engine and emission monitor, one or more engines comprising one or more exhaust pipes, wherein the engine and emission monitor is coupled to the one or more engines, one or more processors operably coupled to the engine and emission monitor, and an exhaust gas analyzer coupled to the one or more exhaust pipes and the engine and emission monitor. The engine and emission monitor may include a plurality of connection ports configured to support a plurality of data communication protocols and a wireless transceiver configured to generate communication links for a long-range wide area network. The exhaust gas analyzer may measure one or more concentration values of one or more gases within an exhaust gas from the one or more engines and transmits the one or more measured concentration values to at least one of the one or more processors. At least one of the one or more processors may cause a stoichiometric model to convert each of the one or more measured concentration values to a mass value using at least an approximate molecular weight of the exhaust gas, a mass value of the exhaust gas, and one or more molecular weights of the one or more gases. The approximate molecular weight of the exhaust gas may be calculated by determining, for each gas of the one or more gases, a product of the measured concentration values of the gas and a molecular weight of the gas, and summing the products for each gas of the one or more gases.

In particular embodiments, the mass value of the exhaust gas is a sum of a mass flowrate of fuel to the one or more engines and a mass flowrate of air to the one or more engines.

In particular embodiments, the mass flowrate of air to the one or more engines is determined by multiplying the mass flowrate of fuel to the one or more engines by an air to fuel ratio.

In particular embodiments, the air to fuel ratio is determined using an oxygen balance or a carbon balance between reactants in air and fuel and reaction products in the exhaust gas.

In particular embodiments, the engine and emission monitor is configured to connect to a plurality of engines, devices, or fuel sensors via the plurality of connection ports.

In particular embodiments, each port in the plurality of connection ports is configured to utilize a different data communication protocol from the plurality of data communication protocols. As an example and not by way of limitation, the plurality of data communication protocols includes one or more of RS422, RS232, CAN BUS, Ethernet/IP, or MODBUS RTU/TCP.

According to another embodiment, a system includes an engine and emission monitor and one or more engines coupled to the engine and emission monitor. The engine and emission monitor may include a plurality of connection ports configured to support a plurality of data communication protocols and a wireless transceiver configured to generate communication links for a long-range wide area network. Each port in the plurality of connection ports may be configured to utilize a different data communication protocol from the plurality of data communication protocols. The engine and emission monitor may be coupled to one or more exhaust pipes of one or more engines. The system further includes one or more processors operably coupled to the engine and emission monitor. The system further includes one or more computer-readable non-transitory storage media including instructions that, when executed by one or more of the processors, cause the one or more of the processors to perform operations. The operations may include detecting an engine type associated with each engine of the one or more engines. The operations may further include automatically selecting, based on the engine type, a data communication protocol from among the plurality of data communication protocols for each engine of the one or more engines. The operations may further include collecting engine data from each engine via a connection port coupled to that engine based on the selected data communication protocol associated with that engine. The operations may further include executing a machine learning model on the collected engine data to predict one or more mass values of one or more gases within an exhaust gas from the one or more engines. The operations may further include adjusting, based on at least one of the predicted mass values, one or more engine parameters associated with the one or more engines.

In particular embodiments, the engine data includes one or more of a fuel consumption, a fuel flowrate, an air flowrate, an engine efficiency, an engine age, a current engine activity, a current engine speed, a current engine state, or an engine percent load.

In particular embodiments, the machine learning model was trained based on a training dataset. The training dataset may include historical engine operation data collected from a plurality of engines during a sampling period. As an example and not by way of limitation, the historical engine operation data includes one or more of a fuel type, a fuel consumption, a fuel flowrate, an air flowrate, an engine efficiency, an engine load, an engine speed, an engine age, or an engine state. The training dataset may further include concentration data of one or more gases in the exhaust gas collected by at least one exhaust gas analyzer during the sampling period. The training dataset may further include ground truth data associated with gases in emissions from the plurality of engines. The ground truth data may be generated based on applying a stoichiometric model to at least a portion of the historical engine operation data and the concentration data of the one or more gases.

In particular embodiments, the sampling period is determined based on one or more of an engine type, an engine state, a fuel type, an operational activity, or an exhaust equipment configuration.

In particular embodiments, the one or more engine parameters is fuel consumption, fuel flowrate, air flowrate, engine efficiency, engine age, current engine activity, current engine state, or engine percent load.

In particular embodiments, the training dataset is updated at a predetermined periodic interval based on changes in one or more of an engine condition, a repair, an overhaul, or an addition of exhaust treatment equipment.

According to another embodiment, a method includes collecting, via one or more connection ports of a plurality of connection ports of an engine and emission monitor, engine data associated with one or more engines. The method further includes inputting the collected engine data set into a machine learning model. The method further includes executing the machine learning model on the collected engine data to predict one or more mass values of one or more gases within an exhaust gas from the one or more engines. The method further includes adjusting, based on at least one of the predicted mass values, one or more engine parameters associated with the one or more engines.

In particular embodiments, the method further includes training the machine learning model based on a training dataset. The training dataset may include historical engine operation data collected from a plurality of engines during a sampling period. As an example and not by way of limitation, the historical engine operation data includes one or more of a fuel type, a fuel consumption, a fuel flowrate, an air flowrate, an engine efficiency, an engine load, an engine speed, an engine age, or an engine state. The training dataset may further include concentration data of one or more gases in the exhaust gas collected by at least one exhaust gas analyzer during the sampling period. The training dataset may further include ground truth data associated with gases in emissions from the plurality of engines. The ground truth data may be generated based on applying a stoichiometric model to at least a portion of the historical engine operation data and the concentration data of the one or more gases.

In particular embodiments, the method further includes updating, at a predetermined periodic interval, the training dataset based on changes in one or more of an engine condition, a repair, an overhaul, or an addition of exhaust treatment equipment.

In particular embodiments, the method further includes transmitting the one or more predicted mass values to a computing system for display.

In particular embodiments, each port in the plurality of connection ports is configured to utilize a different data communication protocol from the plurality of data communication protocols.

In particular embodiments, the method further includes detecting an engine type associated with each engine of the one or more engines. The method further includes automatically selecting, based on the engine type, a data communication protocol from among the plurality of data communication protocols for each engine of the one or more engines. Collecting the engine data from each engine may be based on the data communication protocol associated with that engine.

Advantages of certain embodiments of this disclosure may include one or more of the following. Certain embodiments described herein can provide for more accurate and consistent emissions data by, for example, eliminating periods during which the system is purged and/or eliminating the impact of clogging (e.g., in a manifold connected to the exhaust gas analyzer due to condensation from changing temperatures). Certain embodiments described herein can significantly reduce the cost of purchase of an exhaust gas analyzer and related components and eliminate the time and cost of maintaining the exhaust gas analyzer. Certain embodiments described herein can calculate automated fuel consumption and emission data real-time, which reduces operating expenses and minimizes error. Certain embodiments described herein can optimize the engine loads to reduce the fuel and emissions and identify anomalies which could lead to costly maintenance issues.

Other technical advantages will be readily apparent to one skilled in the art from the following figures, descriptions, and claims. Moreover, while specific advantages have been enumerated above, various embodiments may include all, some, or none of the enumerated advantages.

### EXAMPLE EMBODIMENTS

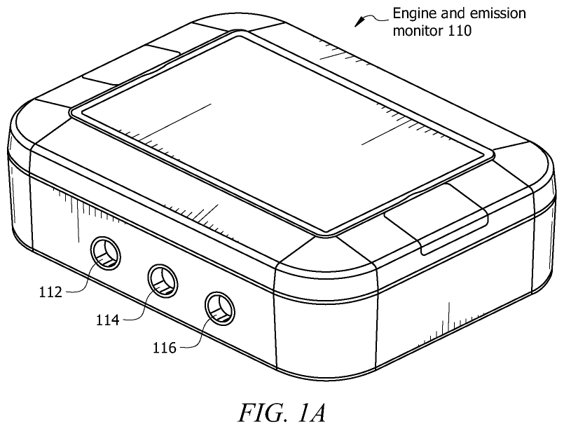

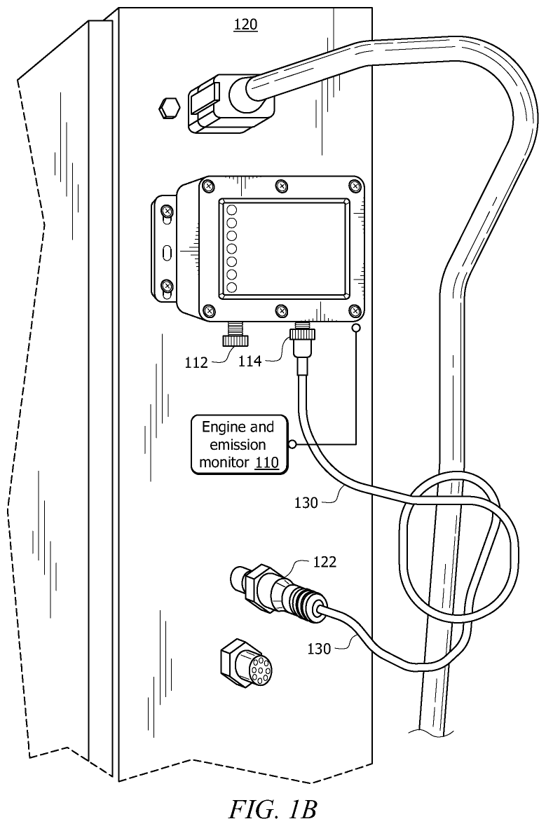

FIG. 1A illustrates an example engine and emission monitor 110. The engine and emission monitor 110 of the present disclosure may include one or more connection ports (e.g., the port 112, the port 114, and the port 116) configured for connections with one or more engines and/or other types of devices. Although FIG. 1A depicts the engine and emission monitor 110 having a particular number of ports, this disclosure contemplates the engine and emission monitor having any suitable number of ports. In some embodiments, the engine and emission monitor 110 may include at least four connection ports. The one or more connection ports may enable the engine and emission monitor 110 to connect to different types of equipment (e.g., commercially available engines, generators, and the like) and collect data therefrom.

The engine and emission monitor 110 may include an enclosure. In some embodiments, the enclosure may be made of plastic (e.g., ABS plastic). In other embodiments, the enclosure may be made of metal (e.g., an explosion-proof metal). In an example embodiment, the dimensions (in millimeters) of the engine and emission monitor 110 may be 90 by 50 by 120. The IP rating of the engine and emission monitor may be IP67. The operating voltage of the engine and emission monitor may be 7-24 VDC powered by engine port. The engine and emission monitor 110 may support communication options including LoRaWAN, WiFi 2.4 Ghz, and Ethernet.

The features of the engine and emission monitor 110 may include web access server for custom configuration, plug-and-play capabilities, multiple backhaul options, lightweight and easy to mount, versatile communication protocols for ECM connection, onboard storage for bulk upload in poor network regions, status LEDs for diagnosis and troubleshooting, and near-real time data acquisition.

FIG. 1B illustrates an example engine and emission monitor 110 connected to an engine 120. As shown in FIG. 1B, the port 114 may be connected to the port 122 of an engine 120 via a cable 130. The engine and emission monitor 110 may connect to various numbers of devices at the same time. Although FIG. 1B depicts that the ports of the engine and emission monitor are used to connect a single engine 120, this disclosure contemplates that the ports of the engine and emission monitor can be used to connect any suitable number of devices at the same time. For example, the engine and emission monitor 110 may connect to two engines and one generator (not shown). As another example, the engine and emission monitor 110 may connect to a device integrating the engine 120 and a generator (not shown). Besides connecting to engines 120, the engine and emission monitor 110 may be connected to fuel sensors, which may be useful when the engine 120 is not an ECMS digital engine 120. In certain embodiments, the engine and emission monitor 110 may collect data from the fuel sensors. For example, the fuel sensor may include an ultrasonic sensor that clamps onto flow pipes and all the data can be collected through a central hub back to the engine and emission monitor 110.

The one or more connection ports may utilize different communication protocols (e.g., Serial RS422 and RS232, CAN BUS, Ethernet/IP, and MODBUS RTU/TCP). In such embodiments, the engine and emission monitor 110 of the present disclosure may include firmware, which may be software that is embedded in the engine and emission monitor and that allows the measurements as described herein. The firmware may include one or more computer-readable non-transitory storage media comprising instructions. When executed by one or more processors operably coupled to the engine and emission monitor 110, the instructions may cause the one or more processors to perform various operations disclosed herein. In particular embodiments, the firmware may enable communications across those different protocols. For example, the engine and emission monitor 110 may receive data from engines using RS485. As another example, the data transmission of standard data between the engine 120 and the engine and emission monitor 110 may be via serial protocols such as J1939, which runs CAN BUS lines. The firmware may have the ability to automatically scan the data transmission to identify what data is available from a particular engine, e.g., when the data transmission is via J1939. The firmware is also adaptable to change based on what engine is connected to the engine and emission monitor 110 by including ability to switch between different protocols. In some embodiments, a user may manually adapt the firmware to switch between different protocols. In other embodiments, the firmware may adapt automatically to switch between different protocols.

In particular embodiments, the one or more processors operably coupled to the engine and emission monitor 110 may include one or more micro processing units (MPUs) or microcontroller units (MCUs) in the engine and emission monitor 110. In particular embodiments, the engine and emission monitor 110 may convert the data collected from engine 120 using its main MPU or MCU, which decodes the data and generates packets for LoRaWAN uplinks. In particular embodiments, a wireless transceiver may be configured to generate communication links for a LoRaWAN. For example, the engine and emission monitor 110 may have a 915-megahertz antenna and a 915-megahertz chip for LoRaWAN, which enables the engine and emission monitor 110 to decode from, e.g., CAN BUS, and convert and uplink via that chip to one or more cloud data lakes 206. The antenna and chip may function as a wireless transceiver.

The engine and emission monitor 110 of the present disclosure may in turn use LoRaWAN technology to communicate with other components of the system (e.g., an Ethernet LAN or WiFi). LoRaWAN is supported by one of the largest networks of IoT devices platform. Data (e.g., engine data and emission measurements) may be secured in a cloud environment, which enables seamless integrations with other operations systems through APIs. In addition, real-time measurements may be available both locally and remotely to minimize potential anomalies and missing data. The machine learning algorithms may include stoichiometric models that convert the actual exhaust into the GHG accurately.

The engine and emission monitor 110 may log engine and gas data every minute. The engine and emission monitor 110 may include a modem and interfaces that collect data using standard communication protocols. The engine and emission monitor 110 may upload the collected data to the cloud from every minute to every hour.

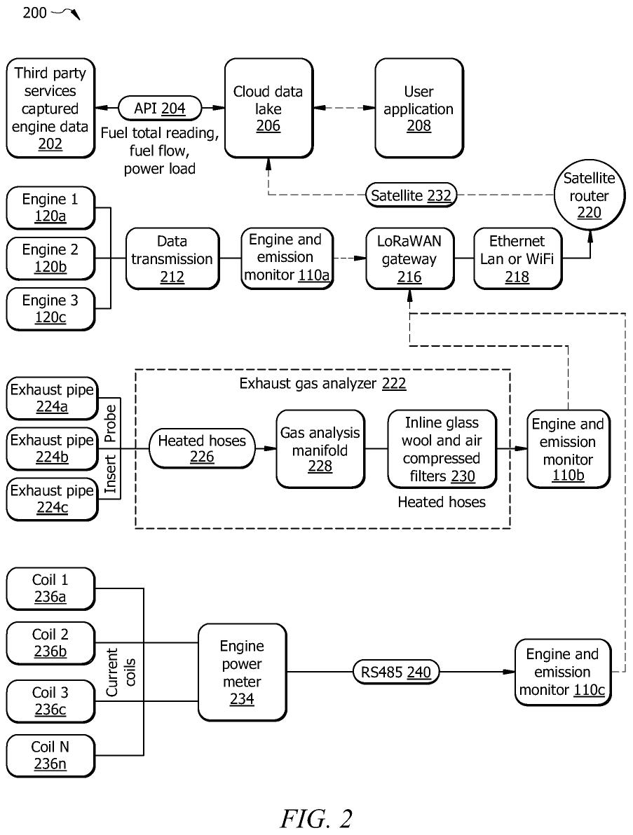

FIG. 2 illustrates an example flow diagram 200 for measuring GHG using the disclosed engine and emission monitor and systems of the present disclosure. The systems of the present disclosure may include one or more engines 120 (120a, 120b, 120c), one or more engine and emission monitors 110 connected to said engines 120, one or more exhaust pipes 224 connected to said engines 120, one or more exhaust gas analyzers 222 connected to said exhaust pipes 224, one or more engine and emission monitors 110 connected to said exhaust gas analyzes 222, one or more coils 236 connect to said engines 120, one or more engine power meter 234 connected to said coils 236, one or more engine and emission monitors 110 connected to said engine power meters 234, one or more LoRaWAN gateways 216 connected to said engine and emission monitors 110, one or more Ethernet LAN or WiFi networks 218 connected to said LoRaWAN gateways 216, one or more satellite routers 220 connected to said Ethernet LAN or WiFi networks 218, one or more satellites 232 connected to said satellite routers 220, one or more cloud data lakes 206 connected to said satellites 232, one or more third party services communicating with said cloud data lakes 206, and one or more user applications 208 communicating with said cloud data lakes 206. Although depicted in FIG. 2 as a single engine and emission monitor, each engine and emission monitor 110a, 110b, and 110c may instead be a plurality of engine and emission monitors. The same is true for the other components of the system depicted in FIGS. 1A-1B (e.g., exhaust gas analyzer 222 and engine power meter 234). In certain embodiments, the one or more exhaust gas analyzers 222 connected to said exhaust pipes 224 and one or more engine and emission monitors 110 connected to said exhaust gas analyzers 222 may be replaced with a virtual gas analyzer that uses a machine learning modeling as described below.

A first engine and emission monitor 110a may receive data transmission 212 from one or more engines 120, e.g., engine 120a, engine 120b, and/or engine 120c. For example, the data transmission 212 may include fuel rate, which indicates how much fuel is being consumed or input into the engine 120. As another example, the data transmission 212 may include air mass, indicating how much air is flowing into the engine 120 overtime. The first engine and emission monitor 110a can collect a plurality of engine pieces data, including but not limited to, total fuel and fuel consumption, predictive diagnostic information such as low oil pressure, coolant temperature or low pressure, power loads and RPM. As will be appreciated by a person or ordinary skill in the art with the benefit of this disclosure, different engines (e.g., from different manufacturers) may have different pieces of data available.

In the illustrated embodiment, an exhaust gas analyzer 222 may collect fuel data such as total fuel consumption, the fuel rate, and the engine percent load. The exhaust gas analyzer 222 may measure any number of gases, such as CO₂, CO, NO, NO₂, SO₂ and CH₄. The exhaust gas analyzer 222 may include sampling probes inserted in the exhaust pipes 224, e.g., exhaust pipe 224a, exhaust pipe 224b, and exhaust pipe 224c. In certain embodiments, the exhaust gas analyzer 222 may include heated hoses 226, gas analysis manifold 228, and inline glass wool and air compressed filters 230. In certain embodiments, the exhaust gas collected from the exhaust pipes 224 may travel through heated hoses 226 and into the gas analysis manifold 228. In some embodiments, the gas analysis manifold 228 may be used to collect emission measurements for multiple engines through a single exhaust gas analyzer 222. In such embodiments, when an engine is not being used, the gas analysis manifold 228 may close a valve (not shown) to eliminate data from that engine being measured by the exhaust gas analyzer 222. The exhaust gas analyzer 222 may be connected to a second engine and emission monitor 110b. In one embodiment, the data from the inline glass wool and air compressed filters 230 may be sent to the second engine and emission monitor 110b. The exhaust gas analyzer 222 can enable direct measurement methods which is usually lower than the calculated emissions. The exhaust gas analyzer 222 can be more accurate than conversion tables.

In the illustrated embodiment, an engine power meter 234 may connect to one or more coils 236, e.g., coil 236a, coil 236b, coil 236c, and coil 236d, directly to the engines 120 or the generators load side. The number of coils may vary from about 1 coil to about 18 coils. The engine power meter 234 may collect, for example, active power data (such as active kWh), L-L or L-N voltage, average power factor, and average current. The engine power meter 234 may function as an integrator integrating data from the coils 236. The engine power meter 234 may be connected to a third engine and emission monitor 110c via RS485 240.

The data collected by the engine and emission monitors 110, the exhaust gas analyzer 222, and the engine power meter 234 may all be sent, via the LoRaWAN gateway 216 of the engine and emission monitors 110, to an Ethernet LAN or WiFi network 218. The Ethernet LAN or WiFi network 218 may further send the data to a satellite router 220, which may send the data to a cloud data lake 206 via a satellite 232.

In particular embodiments, third party services captured engine data 202 such as fuel total reading, fuel flow, power load, may be transmitted to the cloud data lake 206 via one or more application programming interfaces (APIs) 204.

In particular embodiments, the cloud data lake 206 may include a computing system (e.g., server). The computing system may be associated with an entity that provisions the engine and emission monitor 110. In such embodiments, the one or more processors operably coupled to the engine and emission monitor 110 may be part of a computing system in the cloud data lake 206.

In particular embodiments, a user application 208 may access the data from the cloud data lake 206. For example, the user application 208 may include customized dashboards. Data may be accessible in CSV/PDF formats to import into other operational systems. In particular embodiments, a user can use the user application 208 to fill in fuel type, engine efficiency, and engine state (e.g., warming, steady or shutting down). In certain embodiments, the engine state may be determined based at least in part of engine load and/or engine speed.

In particular embodiments, a user can log into the engine and emission monitor 110 over a web access portal (e.g., graphical user interface (GUI)) on the engine and emission monitor 110 itself. The user can change configurations of the engine and emission monitor 110 such as whether it is connected to an engine, type of engine, whether it is connected to the fuel sensors, or whether it is connected to the power meter. The web access portal may also help assess what type of data are available. The web access portal may additionally monitor alerts like engine disconnection or signal degradation. The web access portal may also be used to configure the general settings and communication parameters of the engine and emission monitor 110.

The engine and emission monitor 110 may have one or more of the following features. In one feature, the engine and emission monitor 110 may link engine data to other machines, for example, but not limited to, pumps, rotating equipment, drilling equipment, transportation equipment, and the link. In another feature, the engine and emission monitor 110 may utilize LoRaWAN for standardized, easy-to-install IoT connectivity which seamlessly integrates with thousands of other sensors. In another feature, data may be streamed through an IoT platform in real time. In another feature, cloud-based databases may allow APIs 204 to support other operational software. In another feature, the engine and emission monitor 110 may have access to a plurality of engine diagnostic codes for seamless integrations with preventive maintenance systems. In another feature, the engine and emission monitor 110 may have access through Wi-Fi, Ethernet, cellular, satellite, or Bluetooth Low Energy (BLE).

In particular embodiments, data collected by the one or more engine and emission monitors 110 may be enhanced with stoichiometric or other reaction models and/or artificial intelligence (AI) capabilities, such as machine learning modeling. In certain embodiments, the exhaust gas analyzer 222 may measure concentration (e.g., percentages and/or parts per million (ppm)) values of one or more gases (e.g., greenhouse gases) contained with an exhaust gas from, e.g., an engine. Such gases may include, but are not limited to, carbon dioxide (CO₂), carbon monoxide (CO), water (H₂O), nitric oxide (NO), nitrogen dioxide (NO₂), oxygen (O₂), one or more hydrocarbons (HC) (e.g., methane (CH₄)), nitrogen (N₂), and sulfur dioxide (SO₂). In certain embodiments, those measurements may be sent to the engine and emission monitor 110b. In certain embodiments, stoichiometric models may be used to convert the measured concentration values of the one or more gases to a mass value (e.g., pounds or pounds/minute) of that gas. The conversion may occur on a set time interval. In some embodiments, the conversion may occur every minute. In addition to the measured concentration values of the gases, inputs for the stoichiometric models may further include the data from the engine and emission monitor 110a that receives data from the one or more engines 120. Such data may include, for example, but without limitation, the total fuel consumption, fuel flowrate, and air flowrate. Other inputs to the stoichiometric models may include static variables, such as air density and conversion constants. Such static variables may come from the engine and emission monitor 110 and/or may be input by a user or operator. The stoichiometric models may be run in various locations within the system of in the present disclosure, for example, on engine and emission monitor 110a and/or cloud data lake 206. In some embodiments, the MPU/MCU of the engine and emission monitor 110 can execute the stoichiometric models locally. In some other embodiments, the computing system of the cloud data lake 206 can execute the stoichiometric models remotely.

The details of stoichiometric models according to certain embodiments of the present disclose are described below. In certain embodiments, the stoichiometric model may depend in part on the type of fuel, such as diesel fuel, natural gas, bunker fuel, ammonia, or other biofuels. In certain embodiments, the mass value of a gas (Mass_{G}) (e.g., pounds per minute) in the exhaust gas from the one or more engines may be calculated based on the measured concentration of that gas (Conc_{G}), the molecular weight of that gas (MW_{G}) (e.g., volume percentage), the approximate molecular weight of the exhaust gas (MW_{E}), and the mass value of the exhaust gas (Mass_{E}) (e.g., pounds per minute). As will be appreciated by a person of ordinary skill in the art with the benefit of the present disclosure, the molecular weight of a known gas (e.g., CO₂) may be calculated by summing the molar mass of the individual atoms that make up the gas. In certain embodiments, the mass value may be calculated using the following equation:

$${Mass}_{G} = \frac{C o n {c}_{G}}{100} ⋆ \frac{{MW}_{G}}{{MW}_{E}} * {Mass}_{E} \qquad ( 1 )$$

For the exhaust gas, the exact atoms and quantity of atoms may not be known. As such, an approximate molecular weight of the exhaust gas (MW_{E}) may be used instead. In certain embodiments, the approximate molecular weight of the exhaust gas (MW_{E}) may be determined by summing the products of the measured concentration values (e.g., percentage and/or ppm) of each of the one or more gases (e.g., CO₂, CO, H₂O, NO, NO₂, SO₂, O₂, HC, CH₄, and N₂) and the molecular weight (MW) of that gas. In such embodiments, it may be assumed that the volumetric concentration of each gas is equal to the mole fraction of each gas under ideal gas behavior, as will be appreciated by a person of ordinary skill in the art with the benefit of this disclosure. In certain embodiments, the approximate molecular weight of the exhaust gas may be calculated using the following equation:

$${MW}_{E} = ( \frac{C {O}_{2}}{100} ⋆ {MW}_{{CO}_{2}} ) + ( \frac{C O}{100} ⋆ {MW}_{CO} ) + ( \frac{{H}_{2} O}{100} ⋆ {MW}_{{H}_{2} O} ) + ( \frac{N O}{100} ⋆ {MW}_{NO} ) + ( \frac{N {O}_{2}}{100} ⋆ {MW}_{{NO}_{2}} ) ( \frac{S {O}_{2}}{100} ⋆ {MW}_{{SO}_{2}} ) + ( \frac{{O}_{2}}{100} ⋆ {MW}_{{O}_{2}} + ( \frac{H C}{100} ⋆ {MW}_{HC} ) \begin{matrix}+ & ( \frac{C {H}_{4}}{100} ⋆ {MW}_{{CH}_{2}} ) + ( \frac{{N}_{2}}{100} ⋆ {MW}_{{N}_{2}} )\end{matrix} \qquad ( 2 )$$

In certain embodiments, one or more of the gases may be omitted from the above equation depending, for example, on which gases are measured, the concentration of each gas, and the like. For instance, if the concentration of a gas in the exhaust is low relative to the concentrations of the other gases, it may be excluded when calculating the approximate molecular weight of the exhaust. In certain embodiments, the measured hydrocarbon gas (HC) may be a mixture of hydrocarbons and the exact molecular weight may be unknown. In such embodiment, the stoichiometric model may use an approximate molecular weight of the fuel as the molecular weight of the measured hydrocarbon gas. As will be appreciated by a person of ordinary skill in the art with the benefit of this disclose, the molecular weight of the fuel will vary depending upon, for example, the composition of the fuel.

In certain embodiments, the exhaust gas analyzer 222 may not measure a concentration value of all gases contained within the exhaust gas, for example, water and nitrogen. In such embodiments, the stoichiometric model may estimate the concentration value of those gases. For example, in certain embodiments, the concentration values of water (H₂O) and/or hydrogen (H₂) may be estimated based on the concentration values of carbon dioxide (CO₂) and carbon monoxide (CO) and a water gas shift equilibrium constant. As will be appreciated by a person of ordinary skill in the art with the benefit of this disclose, the water gas shift equilibrium constant may vary depending upon, for example, the composition of the fuel. In certain embodiments, the water gas shift equilibrium constant may be from about 3.5 to about 4.5. In some embodiments, the water gas shift equilibrium constant may be from about 3.6 to about 4.2. In other embodiments, the water gas shift equilibrium constant may be from about 3.5 to about 3.8. In certain embodiments, the stoichiometric model may assume that the balance of the unmeasured gas in the exhaust gas is nitrogen (N₂).

In certain embodiments, the mass value of the exhaust gas (Mass_{E}) may be a mass flowrate (e.g., pounds per minute). In certain embodiments, the mass flowrate of the exhaust gas may be the sum of the mass flowrate of fuel and the mass flowrate of air. In certain embodiments, the flowrate of the fuel may be measured, for example, by the engine itself or using one or more sensors on the input and return fuel lines. In some embodiments, the mass flowrate of the fuel may be measured. In other embodiments, the volumetric flowrate of the fuel may be measured and converted to a mass flowrate of the fuel using the density (or approximate density) of the fuel. In certain embodiments, the flowrate of the fuel may be measured, for example, by the engine itself or using one or more exhaust probes (e.g., as part of the exhaust gas analyzer described herein). In some embodiments, the mass flowrate of air may be measured. In other embodiments, the volumetric flowrate of air may be measured and converted to a mass flowrate of air using the density (or approximate density) of the air. In other embodiments, the flowrate of air may be determined by multiplying the mass flowrate of fuel by a calculated air to fuel ratio (A/F).

In certain embodiments, the air to fuel (A/F) ratio may be calculated using an oxygen balance, a carbon balance, or both. As will be appreciated by a person of ordinary skill in the art with the benefit of this disclosure, an oxygen balance balances oxygen atoms in the reactants (e.g., air and fuel) with oxygen atoms in the reaction products (e.g., exhaust gases). Using an oxygen balance, the air to fuel (A/F) ratio may be calculated by multiplying: (i) a conversion constant by (ii) the ratio of the molecular weight of air (Mₐᵢᵣ) to the molecular weight of fuel (M_{fuel}) by (iii) the ratio of the mole fractions of oxygen-containing species in the exhaust to the mole fractions of carbon-containing species in the exhaust. In certain embodiments, the conversion constant may be based on the number of moles of air per mole of oxygen. In some embodiments, it may be assumed that air is composed of only nitrogen (N₂) and oxygen (O₂) and that there are 3.773 moles of nitrogen (N₂) and oxygen (O₂), which approximately translates to 79% nitrogen (N₂) and 21% oxygen (O₂). In such embodiments, the conversion constant may be 4.773. In certain embodiments, the correction factor may be used to adjust for a lean environment (e.g., excess air relative to fuel) or a rich environment (e.g., excess fuel relative to air). In certain embodiments, the air to fuel (A/F) ratio may be calculated using the following equation:

$$\frac{A}{F} = 4.773 ( \frac{{MW}_{air}}{{MW}_{f}} ) \frac{( {CO}_{2} ) + \frac{( CO )}{2} + \frac{( {H}_{2} O )}{2} + \frac{( NO )}{2} + ( {NO}_{2} ) + ( {O}_{2} )}{( HC ) + ( CO ) + ( {CO}_{2} )} \qquad ( 3 )$$

As will be appreciated by a person of ordinary skill in the art with the benefit of this disclosure, a carbon balance balances carbon atoms in the reactants (e.g., air and fuel) with carbon atoms in the reaction products (e.g., exhaust gases). Using a carbon balance, the air to fuel (A/F) ratio may be calculated by multiplying: (i) the ratio of the molecular weight of air (Mₐᵢᵣ) to the molecular weight of fuel (M_{fuel}) by (ii) a correction factor. In certain embodiments, the correction factor may be used to adjust for a lean environment (e.g., excess air relative to fuel) or a rich environment (e.g., excess fuel relative to air). In certain embodiments, the air to fuel (A/F) ratio may be calculated using the following equation:

$$\frac{A}{F} = ( \frac{{MW}_{air}}{{MW}_{f}} ) [ \frac{100 + ( HC ) - \frac{( CO )}{2} + \frac{3 ( {H}_{2} O )}{2} - {H}_{2} {O}_{n}}{( H C ) + ( CO ) + ( {CO}_{2} )} - \frac{y}{2} ] \qquad ( 4 )$$

In the above equation, H₂Oα is the volume or molar percent water vapor as measured at the analyzer(s) measuring exhaust gas. In certain embodiments, H₂Oα may range from about 0% to about 2%. In other embodiments, H₂Oα may range from about 0% to about 1%. In the above equation, y is the ratio of hydrogen to carbon in the fuel. In certain embodiments, y may range from about 1 (e.g., for methane (CH₄)) to about 4. In some embodiments, y may range from about 1 to about 3. In other embodiments, y may range from about 1 to about 2. In certain embodiments, the air to fuel (A/F) ratio calculated using an oxygen balance may be the identical or nearly identical to the air to fuel (A/F) ratio calculated using a carbon balance.

In particular embodiments, a machine learning model may be used as a virtual gas analyzer in place of the exhaust gas analyzer 222 and the engine and emission monitor 110b. Once the machine learning model is developed, the use of an exhaust gas analyzer 222 as described above would be only a temporary installation to train the machine learning model as discussed below, and therefore, a single exhaust gas analyzer 222 (or some number of exhaust gas analyzers 222 less than the number of engines 120) to collect samples of engine and exhaust data to build machine learning models that could be used to support a large fleet, rig, etc., which would in turn eliminate the need of a permanent installation of a gas analyzer on the total fleet thereby reducing a significant expense.

Among the many potential benefits and advantages of the virtual gas analyzer of the present disclosure, only some of which are alluded to herein, the virtual gas analyzer and its use of a machine learning model meet the International Maritime Organization (IMO) requirements for calculating emissions. They further may offer higher accuracy compared to traditional calculation methods (e.g., using conversion tables) by using, for example, data from specific engine models, states, and fuel types. Furthermore, in certain embodiments, the emissions calculations may be automated and/or embedded into existing operations and regulatory reports.

The machine learning model may be, for example, and without limitation, a linear regression model or a proprietary model developed based on training data. In some embodiments, the machine learning modeling can incorporate models in open code. The computing system of the cloud data lake 206 may apply the machine learning model to input data from one or more engine and emission monitors (e.g., 110a), each connected to one or more engines 120 or other equipment, and/or data input manually by a user or operator. Such input data may include, for example, fuel data, fuel consumption, fuel flowrate, air flowrate, the efficiency of the engine, the age of the engine, the current activity of the engine, the current state of the engine, and/or the percent load of the engine. The output of the machine learning model to mass values of one or more exhaust gases as described above as the output from the stoichiometric models of the present disclosure.

To train the machine learning model, particular embodiments may use an exhaust gas analyzer 222 to collect and sample exhaust data as described above during the sampling period. In particular embodiments, the training data for training the machine learning model may include historical engine operation data collected from one or more engine and emission monitors 110a-c a particular fleet or rig as described above and concentration data of one or more gases in the exhaust gas collected by at least one exhaust gas analyzer as described above. Such data may be considered the ground truth. In some embodiments, a stoichiometric model of the present disclosure may be applied to the collected data to generate mass values of one or more exhaust gases for a particular fuel as described above. In such embodiments, the generated mass values may be considered the ground truth. However, it may be costly and time-consuming to obtain sufficient amount of historical data. Alternatively, the training data may include current running data. In such embodiments, one or more portable or temporary exhaust gas analyzer 222 can be installed on fleet or rig and collect training data for a period of time. The period of time may be based on predetermined factors, including but not limited to, the type of engines, engine states, types of fuels, operational activities and vessel's exhaust equipment. In certain embodiments, the period of time for which training data is collected may be up to one month. In other embodiments, the period of time for which training data is collected may be up to two weeks. In yet other embodiments, the period of time for which training data is collected may be from about 24 hours to about 1 week. The portable or temporary exhaust gas analyzer 222 can be reused across multiple engines and/or fleets/rigs to collect data over a larger period of time. In certain embodiments, the period of time in which the portable or temporary gas analyzer 222 is install is shorted than the overall period of time for which the particular engines or fleet/rig are expected to run. In certain embodiments, the training data can be re-tuned at a certain frequency (e.g., every 6 months to a year) to maintain accuracy. Re-tuning data may be especially important after any engine repair or overhauls or additions of equipment (e.g., scrubbers or similar modules) in the system. The machine learning model can then be trained for a specific engine, fleet/rig, and/or fuel instead of relying on historical data that may be from different sources (e.g., engines, fuel, etc.). As a result, the machine learning model may be more accurate using current data as opposed to historic data.

The collected sample exhaust data as described above during the sampling period may be associated with a specific fuel type. As such, the machine-learning model trained using such sample exhaust data may be used for the corresponding fuel type. Sample exhaust data for different fuel types may be collected to train multiple machine-learning models, with each appliable to a specific fuel type among the different fuel types. When being used for prediction of mass values of gases, a user may specify the fuel type and the embodiments disclosed herein may select the machine-learning model trained for that fuel type and use the selected machine-learning model for prediction.

The machine learning model may learn different engine states, different engine percent loads, different fuel rates, and/or the percentages of gas getting out of the engines 120. The machine learning model can then be used to predict the gas emissions.

The machine learning model may be a supervised machine learning model that trains algorithms on labeled datasets to make predictions and/or learns a relationship between inputs and corresponding outputs. The machine learning model may be known model type, e.g., a linear regression model, or may be a proprietary model developed based on training data as would be appreciated by a person of ordinary skill in the art with the benefit of this disclosure. In certain embodiments, the input variables (e.g., ratio of engine state unloading versus percentage output) may be different from engine 120 to engine 120. Such input variables may be collected specifically for an engine 120, for which the machine learning model can effectively predict the mass of one or more exhaust gases for that engine 120.

In particular embodiments, replacing the exhaust gas analyzer 222 with a machine learning model may require a replacement of the actual percentage values. The machine learning model can predict the actual percentage values based on current state of the engine 120. Depending on how heavy the engine 120 is running, and the percent load is of that engine 120, the machine learning model can determine the percentage of gas in the exhaust.

The machine learning model and stoichiometric models may be both used to calculate and/or predict the mass of one or more exhaust gases. In certain embodiments, the machine learning model and stoichiometric models may use different input data, but they would each produce the similar results (e.g., mass of one or more gasses in the emission). In particular embodiments, the machine learning model may be used to predict the mass of one or more exhaust gases based on engine data collected from engines. In this case, neither the exhaust gas analyzer nor the stoichiometric models are required. The machine learning model can integrate the functions of the exhaust gas analyzer and the stoichiometric models. In some embodiments, the machine learning model may be used in combination with the stoichiometric models to predict the mass of one or more exhaust gases. In this case, the machine learning model may replace the exhaust gas analyzer. The machine learning model may predict concentrations of gases within an exhaust gas from engines based on engine data collected from the engines. The output from the machine learning model may then be provided to one or more stoichiometric models to predict the mass of one or more gases within the exhaust gas.

The virtual gas analyzer may increase the accuracy of the calculated method traditionally used in the industry because machine learning model may incorporate fuel type or composition and engine parameters (e.g., engine state, load, etc.). In addition, the emission measurements collected from the virtual gas analyzer may be more reliable and accurate since it may be used to eliminate the impact of inaccurate and/or inapplicable data during period of purging and/or clogging. Furthermore, the virtual gas analyzer may reduce capital and operating expenses since it eliminates the need of installing an exhaust gas analyzer for each engine.

In particular embodiments, the data collected by the engine and emission monitor 110 and/or calculated using the systems and methods of present disclosure may be displayed on one or more dashboards.

In particular embodiments, the output from the machine learning model (either alone or in combination with the one or more stoichiometric models) may be used to perform various actions responsive to the predicted mass values of gases within an exhaust gas from the one or more engines. In certain embodiments, the output from the machine learning model (either alone or in combination with the one or more stoichiometric models) may be used to adjust one or more parameters associated with the one or more engines from which the exhaust gas is emitted. In some embodiments, a human may manually adjust one or more parameters associated with the engines based on the output from the machine learning model (either alone or in combination with the one or more stoichiometric models). In such embodiments, the MPU/MCU of the engine and emission monitor 110 or the computing system of the cloud data lake 206 may send instructions to a human to adjust the engines to adjust the parameters. Such parameters associated with the engines that may be adjusted based on the output from the machine learning model (either alone or in combination with the one or more stoichiometric models) include, but are not limited to, fuel consumption, fuel flowrate, engine speed, current engine activity, current engine state, engine percent load, and the like. In certain embodiments, one or more engine and/or one or more parts thereof (e.g., filter) may be replaced based on the output from the machine learning model (either alone or in combination with the one or more stoichiometric models). As a result of such adjustments and/or replacements, the amount of fuel being burned by the one or more engines may be reduced, which, in turn, may reduce emissions and/or fuel costs.

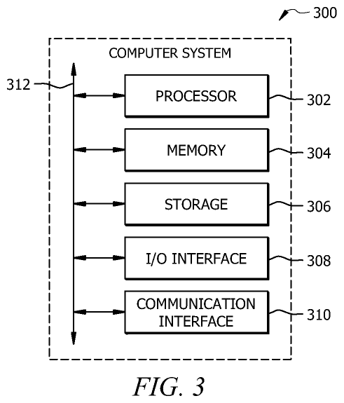

FIG. 3 illustrates an example computer system 300. In particular embodiments, one or more computer system 300 perform one or more steps of one or more methods described or illustrated herein. In particular embodiments, one or more computer system 300 provide functionality described or illustrated herein. In particular embodiments, software running on one or more computer system 300 performs one or more steps of one or more methods described or illustrated herein or provides functionality described or illustrated herein. Particular embodiments include one or more portions of one or more computer system 300. Herein, reference to a computer system may encompass a computing device, and vice versa, where appropriate. Moreover, reference to a computer system may encompass one or more computer systems, where appropriate.

This disclosure contemplates any suitable number of computer system 300. This disclosure contemplates computer system 300 taking any suitable physical form. As example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 300 may include one or more computer system 300; be unitary or distributed; span multiple locations; span multiple machines; span multiple data centers; or reside in a cloud, which may include one or more cloud components in one or more networks. Where appropriate, one or more computer system 300 may perform without substantial spatial or temporal limitation one or more steps of one or more methods described or illustrated herein. As an example, and not by way of limitation, one or more computer system 300 may perform in real time or in batch mode one or more steps of one or more methods described or illustrated herein. One or more computer system 300 may perform at different times or at different locations one or more steps of one or more methods described or illustrated herein, where appropriate.

In particular embodiments, computer system 300 includes a processor 302, a memory 304, a storage 306, an input/output (I/O) interface 308, a communication interface 310, and a bus 312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 302 includes hardware for executing instructions, such as those making up a computer program. As an example and not by way of limitation, to execute instructions, processor 302 may retrieve (or fetch) the instructions from an internal register, an internal cache, memory 304, or storage 306; decode and execute them; and then write one or more results to an internal register, an internal cache, memory 304, or storage 306. In particular embodiments, processor 302 may include one or more internal caches for data, instructions, or addresses. This disclosure contemplates processor 302 including any suitable number of any suitable internal caches, where appropriate. As an example and not by way of limitation, processor 302 may include one or more instruction caches, one or more data caches, and one or more translation lookaside buffers (TLBs). Instructions in the instruction caches may be copies of instructions in memory 304 or storage 306, and the instruction caches may speed up retrieval of those instructions by processor 302. Data in the data caches may be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data. The data caches may speed up read or write operations by processor 302. The TLBs may speed up virtual-address translation for processor 302. In particular embodiments, processor 302 may include one or more internal registers for data, instructions, or addresses. This disclosure contemplates processor 302 including any suitable number of any suitable internal registers, where appropriate. Where appropriate, processor 302 may include one or more arithmetic logic units (ALUs); be a multi-core processor; or include one or more processors 302. Although this disclosure describes and illustrates a particular processor, this disclosure contemplates any suitable processor.

In particular embodiments, memory 304 includes main memory for storing instructions for processor 302 to execute or data for processor 302 to operate on. As an example and not by way of limitation, computer system 300 may load instructions from storage 306 or another source (such as, for example, another computer system 300) to memory 304. Processor 302 may then load the instructions from memory 304 to an internal register or internal cache. To execute the instructions, processor 302 may retrieve the instructions from the internal register or internal cache and decode them. During or after execution of the instructions, processor 302 may write one or more results (which may be intermediate or final results) to the internal register or internal cache. Processor 302 may then write one or more of those results to memory 304. In particular embodiments, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere). One or more memory buses (which may each include an address bus and a data bus) may couple processor 302 to memory 304. Bus 312 may include one or more memory buses, as described below. In particular embodiments, one or more memory management units (MMUs) reside between processor 302 and memory 304 and facilitate accesses to memory 304 requested by processor 302. In particular embodiments, memory 304 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 304 may include one or more memories 304, where appropriate. Although this disclosure describes and illustrates particular memory, this disclosure contemplates any suitable memory.

In particular embodiments, storage 306 includes mass storage for data or instructions. As an example and not by way of limitation, storage 306 may include a hard disk drive (HDD), a floppy disk drive, flash memory, an optical disc, a magneto-optical disc, magnetic tape, or a Universal Serial Bus (USB) drive or a combination of two or more of these. Storage 306 may include removable or non-removable (or fixed) media, where appropriate. Storage 306 may be internal or external to computer system 300, where appropriate. In particular embodiments, storage 306 is non-volatile, solid-state memory. In particular embodiments, storage 306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form. Storage 306 may include one or more storage control units facilitating communication between processor 302 and storage 306, where appropriate. Where appropriate, storage 306 may include one or more storages 306. Although this disclosure describes and illustrates particular storage, this disclosure contemplates any suitable storage.

In particular embodiments, I/O interface 308 includes hardware, software, or both, providing one or more interfaces for communication between computer system 300 and one or more I/O devices. Computer system 300 may include one or more of these I/O devices, where appropriate. One or more of these I/O devices may enable communication between a person and computer system 300. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 308 for them. Where appropriate, I/O interface 308 may include one or more device or software drivers enabling processor 302 to drive one or more of these I/O devices. I/O interface 308 may include one or more I/O interfaces 308, where appropriate. Although this disclosure describes and illustrates a particular I/O interface, this disclosure contemplates any suitable I/O interface.

In particular embodiments, communication interface 310 includes hardware, software, or both providing one or more interfaces for communication (such as, for example, packet-based communication) between computer system 300 and one or more other computer system 300 or one or more networks. As an example and not by way of limitation, communication interface 310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 310 for it. As an example and not by way of limitation, computer system 300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 300 may include any suitable communication interface 310 for any of these networks, where appropriate. Communication interface 310 may include one or more communication interfaces 310, where appropriate. Although this disclosure describes and illustrates a particular communication interface, this disclosure contemplates any suitable communication interface.

In particular embodiments, bus 312 includes hardware, software, or both coupling components of computer system 300 to each other. As an example and not by way of limitation, bus 312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 312 may include one or more buses 312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

### EXAMPLES

To facilitate a better understanding of the present disclosure, the following example of certain aspects of certain embodiments are given. The following example is not the only example that could be given according to the present disclosure and is not intended to limit the scope of the disclosure or claims.

A field test was conducted for about five months in mid- to late-2023 with five Caterpillar 3516C engines running on diesel fuel with no additives or blends on a rig. Emissions calculations were made every minute using: (1) the embodiments of the present disclosure with a gas analyzer and a stoichiometric model as described above and (2) the actual fuel consumption and a standard conversion factor table published by the varies regulatory bodies (e.g., EPA). In the field test, the emissions calculated using the embodiments of the present disclosure were lower than those calculated using the traditional method based on actual fuel consumption and conversion tables. On average, the total emissions determined using the embodiments disclosed herein during the period of trustworthy data were 2.97% less than those calculated using the standard conversion factor table published by the EPA. On a few individual days, the total emissions determined using the embodiments disclosed herein were 10%-14% less than those calculated using the traditional method.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Additionally, although this disclosure describes or illustrates particular embodiments as providing particular advantages, particular embodiments may provide none, some, or all of these advantages.

The embodiments disclosed herein are only examples, and the scope of this disclosure is not limited to them. Particular embodiments may include all, some, or none of the components, elements, features, functions, operations, or steps of the embodiments disclosed herein. Embodiments disclosed herein include methods, apparatuses, and systems, wherein any feature mentioned in one category, e.g., a method, can be applied in another category, e.g., a system, as well.

1. A system, comprising:
an engine and emission monitor comprising:
a plurality of connection ports configured to support a plurality of data communication protocols; and
a wireless transceiver configured to generate communication links for a long-range wide area network;
one or more processors operably coupled to the engine and emission monitor; and
one or more engines comprising one or more exhaust pipes, wherein the engine and emission monitor is coupled to the one or more engines;
an exhaust gas analyzer coupled to the one or more exhaust pipes and the engine and emission monitor;
wherein the exhaust gas analyzer measures one or more concentration values of one or more gases within an exhaust gas from the one or more engines and transmits the one or more measured concentration values to at least one of the one or more processors;
wherein at least one of the one or more processors cause a stoichiometric model to convert each of the one or more measured concentration values to a mass value using at least an approximate molecular weight of the exhaust gas, a mass value of the exhaust gas, and one or more molecular weights of the one or more gases; and
wherein the approximate molecular weight of the exhaust gas is calculated by determining, for each gas of the one or more gases, a product of the measured concentration values of the gas and a molecular weight of the gas, and summing the products for each gas of the one or more gases.

2. The system of claim 1, wherein the mass value of the exhaust gas is a sum of a mass flowrate of a fuel to the one or more engines and a mass flowrate of air to the one or more engines.

3. The system of claim 2, wherein the mass flowrate of air to the one or more engines is determined by multiplying the mass flowrate of fuel to the one or more engines by an air to fuel ratio.

4. The system of claim 3, wherein the air to fuel ratio is determined using an oxygen balance or a carbon balance between reactants in air and the fuel and reaction products in the exhaust gas.

5. The system of claim 1, wherein the engine and emission monitor is configured to connect to a plurality of engines, devices, or fuel sensors via the plurality of connection ports.

6. The system of claim 1, wherein each port in the plurality of connection ports is configured to utilize a different data communication protocol from the plurality of data communication protocols, and wherein the plurality of data communication protocols comprises one or more of RS422, RS232, CAN BUS, Ethernet/IP, or MODBUS RTU/TCP.

7. A system, comprising:
an engine and emission monitor comprising:
a plurality of connection ports configured to support a plurality of data communication protocols, wherein each port in the plurality of connection ports is configured to utilize a different data communication protocol from the plurality of data communication protocols; and
a wireless transceiver configured to generate communication links for a long-range wide area network;
one or more engines coupled to the engine and emission monitor;
one or more processors operably coupled to the engine and emission monitor; and
one or more computer-readable non-transitory storage media comprising instructions that, when executed by one or more of the processors, cause the one or more of the processors to perform operations comprising:
detecting an engine type associated with each engine of the one or more engines;
selecting, based on the engine type, a data communication protocol from among the plurality of data communication protocols for each engine of the one or more engines;
collecting engine data from each engine via a connection port coupled to that engine based on the selected data communication protocol associated with that engine;
executing a machine learning model on the collected engine data to predict one or more mass values of one or more gases within an exhaust gas from the one or more engines; and
adjusting, based on at least one of the predicted mass values, one or more engine parameters associated with the one or more engines.

8. The system of claim 7, wherein the engine data comprises one or more of a fuel consumption, a fuel flowrate, an air flowrate, an engine efficiency, an engine age, a current engine activity, a current engine speed, a current engine state, or an engine percent load.

9. The system of claim 7, wherein the machine learning model was trained based on a training dataset comprising:
historical engine operation data collected from a plurality of engines during a sampling period, wherein the historical engine operation data comprises one or more of a fuel consumption, a fuel flowrate, an air flowrate, an engine efficiency, an engine load, an engine speed, an engine age, or an engine state;
concentration data of one or more gases in the exhaust gas collected by at least one exhaust gas analyzer during the sampling period; and
ground truth data associated with gases in emissions from the plurality of engines, wherein the ground truth data is generated based on applying a stoichiometric model to at least a portion of the historical engine operation data and the concentration data of the one or more gases.

10. The system of claim 9, wherein the sampling period is determined based on one or more of an engine type, an engine state, a fuel type, an operational activity, or an exhaust equipment configuration.

11. The system of claim 9, wherein the training dataset is updated at a predetermined periodic interval based on changes in one or more of an engine condition, a repair, an overhaul, or an addition of exhaust treatment equipment.

12. The system of claim 7, wherein the one or more engine parameters is fuel consumption, fuel flowrate, air flowrate, engine efficiency, engine age, current engine activity, current engine state, or engine percent load.

13. A method comprising:
collecting, via one or more connection ports of a plurality of connection ports of an engine and emission monitor, engine data associated with one or more engines;
inputting the collected engine data set into a machine learning model;
executing the machine learning model on the collected engine data to predict one or more mass values of one or more gases within an exhaust gas from the one or more engines; and
adjusting, based on at least one of the predicted mass values, one or more engine parameters associated with the one or more engines.

14. The method of claim 13, further comprising:
training the machine learning model based on a training dataset comprising:
historical engine operation data collected from a plurality of engines during a sampling period, wherein the historical engine operation data comprises one or more of a fuel consumption, a fuel flowrate, an air flowrate, an engine efficiency, an engine load, an engine speed, an engine age, or an engine state;
concentration data of one or more gases in the exhaust gas collected by at least one exhaust gas analyzer during the sampling period; and
ground truth data associated with gases in emissions from the plurality of engines, wherein the ground truth data is generated based on applying a stoichiometric model to at least a portion of the historical engine operation data and the concentration data of the one or more gases.

15. The method of claim 14, wherein the sampling period is determined based on one or more of an engine type, an engine state, a fuel type, an operational activity, or an exhaust equipment configuration.

16. The method of claim 14, further comprising:
updating, at a predetermined periodic interval, the training dataset based on changes in one or more of an engine condition, a repair, an overhaul, or an addition of exhaust treatment equipment.

17. The method of claim 13, wherein the one or more engine parameters is fuel consumption, fuel flowrate, air flowrate, engine efficiency, engine age, current engine activity, current engine state, or engine percent load.

18. The method of claim 13, further comprising:
transmitting the one or more predicted mass values to a computing system for display.

19. The method of claim 13, wherein each port in the plurality of connection ports is configured to utilize a different data communication protocol from a plurality of data communication protocols.

20. The method of claim 19, further comprising:
detecting an engine type associated with each engine of the one or more engines; and
selecting, based on the engine type, a data communication protocol from among the plurality of data communication protocols for each engine of the one or more engines;
wherein collecting the engine data from each engine is based on the data communication protocol associated with that engine.
